(19) United States
(12) Patent Application Publication
(10) Pub. No.: US 2010/0234942 A1
(43) Pub. Date: Sep. 16, 2010

(54) TRANSITION LENSES WITH VIRTUAL PUPIL

(76) Inventor: Gholam A. Peyman, Sun City, AZ (US)

Correspondence Address: THOMPSON HINE L.L.P.;Intellectual Property Group, P.O. BOX 8801, DAYTON, OH, 45401-8801, US

(21) Appl. No.: 12/706,369

(22) Filed: Feb. 16, 2010

Related U.S. Application Data
(60) Provisional application No. 61/159,175, filed on Mar. 11, 2009.
(60) Provisional application No. 61/163,369, filed on Mar. 25, 2009.

(51) Int. Cl. A61F 2/16

(52) U.S. Cl. 623/6.17

(57) ABSTRACT

A lens containing a chromophore distributed in or on the lens such that the lens functions as a virtual pupil in adjusting to light. The lens can be intraocular or extraocular.

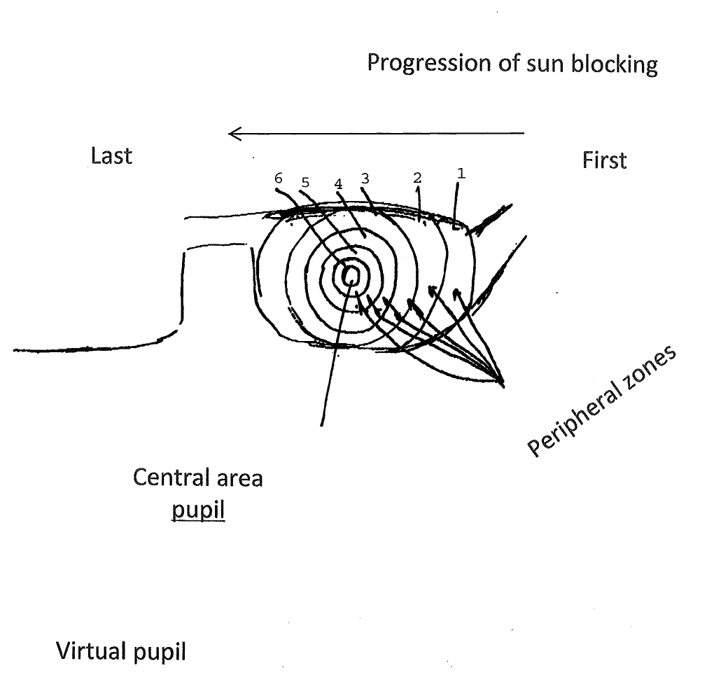

This application claims priority to pending U.S. Application Ser. Nos. 61/163,369 filed Mar. 25, 2009 and 61/159,175 filed Mar. 11, 2009, and expired U.S. Application Ser. No. 61/148,085 filed Jan. 29, 2009.

Intraocular lenses (IOL) are implanted in the eye after removal of a cataract or crystalline lens to replace the diopteric power of the crystalline lens and to focus the light on the retina. IOL can be implanted in the anterior chamber, posterior chamber, in the lens capsule, over another IOL or a normal lens, etc. IOL can correct spheric or astigmatic, etc. problems by altering the refractive power of the eye.

The adjustment ability of IOL can be adjusted after surgery with light, and then stabilized permanently at the desired level. IOL can be monofocal, bifocal, multifocal, etc.

IOL can be configured to contain permanently, ultraviolet (UV) or other wavelength-absorbing pigment (e.g., yellow). The lenses can be made from polymers, e.g., silicone, methacrylates (e.g. (poly)methacrylates), (hydroxyethyl)methacrylate (HEMA), glass, etc. Polymers are known in the art and may be organic, inorganic, or organic and inorganic.

To the inventor's knowledge, until now, there are no IOL such that its pigmentation (light absorbing chemical) can be adjusted automatically by the intensity of the external light to prevent glare. Glare is a recognized disability, e.g. outdoors, or in eyes with minimal or no natural pigment (albinos), or when the iris is damaged or lost after a trauma. Glare reduces visual performance and/or visual acuity because of stray light reducing retinal contrast. To the inventor's knowledge, until now, there are also no IOL or lenses that create a virtual pupil which expands in the dark and constricts in the light, depending on the light intensity.

The FIGURE describes how the inventive lens functions as a virtual pupil. It illustrates the central area, i.e., the pupilary area, and peripheral zones arbitrarily divided in areas 1, 2, 3, 4, 5, 6 for illustrative purposes only. Upon light exposure, zone 1 darkens first and zone 6 darkens last. Zone 1 contains a higher concentration of chromophore than zone 2; zone 2 contains a higher concentration of chromophore than zone 3; zone 3 contains a higher concentration of chromophore than zone 4; zone 4 contains a higher concentration of chromophore than zone 5; and zone 5 contains a higher concentration of chromophore than zone 6. The progression of chromophore concentration from zones 6 to 1 indicate the progression of light block. The central area be one of several embodiment. In one embodiment, the central area contains no chromophore and is transparent. In one embodiment, the central area contains only a chromophore that absorbs UV light. In one embodiment, the central area contains a minimum concentration of chromophore. A minimum concentration is the concentration that will maintain the central area mostly transparent, allowing sight even after a fast transition from light to dark, and achieving minimal darkening of the central area upon exposure to the brightest light.

Although light transitional external glasses exist, to the inventor's knowledge, no one has suggested the use of this technology for intraocular lenses for fear of toxicity and pigment leaking out into the eye. There have been problems with fast adjustment of the chromophore from light medium to dark medium, causing difficulty in vision.

Thus, there is a need to create an IOL for use in animal eye to prevent glare, and that has an automatic adjustable virtual pupil when there is no pupil or in albinos.

Disclosed is a method of creating a virtual pupil for lenses, including lenses used for sunglasses as well as contacts, inlay lens, and IOL.

The method creates, e.g. by coating the lens surface, concentric circles of light-reactive coating material that are centered around the visual axis of the lens. A chromophore that can be activated and is used to coat the lens. Coating refers to both applying the light-activated chromophore to the surface of the lens, and also embedding the light-activated chromophore within the lens material. The coating concentration, e.g. intensity, is increased in these (circular) areas moving from the center toward the periphery of the lens. This embodiment causes the peripheral circle area, upon activation, to become dark first. Activation may be by light (photoactivation), electricity, or other methods. The darkening moves toward the center of the lens as the light intensity increases, thereby creating a virtual pupil that responds to the light intensity similarly to a normal pupil. The central area, which is centered on the visual axis, remains substantially free of these coating (except for prior UV absorber if needed).

Any chemical or a material that can be stimulated by light or electrical potential to change (adjust) its color or pigmentation automatically can be used. The chromophore does not darken the lens permanently. In one embodiment, the chromophore is a photochromic molecule, i.e., a molecule that is activated by light and, upon activation, darkens the lens. Examples of photochromic molecules that are activated by UV light are oxazines and naphthopyrans. An example of photochromic molecules that is activated by visible light is silver chloride. In one embodiment, the photochromic molecule responds to light in the UV and visible spectrum. In one embodiment, the photochromic molecule is silver chloride. In one embodiment, multiple different chromophores are used. In one embodiment, chromophores include, but are not limited to, those that absorb UV light, those that absorb visible light, those that polarize light, and combinations of these.

In one embodiment the lens is extraocular. The lens may be used in contact lenses (e.g., cosmetic, refractive, scleral, bandage, etc.), in glasses, goggles, telescopes, cameras, microscopes, etc. The technology can be used in gear or equipment for all kinds of sports-related indoor or outdoor activity including hunting, golf, tennis, etc. and in activities where goggles, etc are used. In one embodiment, the method may be applied to telescope lens. In one embodiment, the lens is incorporated in clip-on glasses that attach to regular glasses (i.e., spectacles). In one embodiment, the clip-on glasses are themselves sunglasses. In one embodiment, the spectacles are sunglasses.

In one embodiment, the central area of the external lenses is covered, as desired. In one embodiment, the central area is a polymeric material that either lacks or contains a sufficiently low concentration of light-activated chromophore to have substantially no change upon activation. In one embodiment, the central area is a polymeric material that is coated with a chromophore that absorbs light only in the UV region of the electromagnetic spectrum. The rest of the lens is coated with low concentration of light reactive agents, i.e., chromophores. In one embodiment, subsequent coating is repeated while the central covered area is gradually increased. Thus, as more coating is done, more coating will be present in the periphery than the centrally located circles. This permits the glasses to darken gradually from the periphery toward the center as the light intensity increases. In one embodiment, other variations of this concept can be employed.

Any polymeric material (plastics) can be used in conjunction with the chromophore to make a desired IOL with any diopteric specification.

The distribution of the chromophore in the lens (IOL) can be diffuse or local, in the IOL or on its surface.

The distribution of the chromophore (chemical/material) can exclude a portion of the IOL.

The distribution of the chromophore can be mainly in the lens periphery excluding an area (circular or any shape) in the center of the lens or visual axis. This central area can have a diameter of 0.5-5 mm or more (IOL ideal 1-2 mm, contact lens 1-4 mm, glasses 2-5 mm or more) so that the light passes through this area of the lens is not impeded or absorbed by the chromophore or light absorbing material.

There can be a gradual transition area between areas containing the chromophore and areas lacking the chromophore. The degree of achieved darkening (or change in color) depends on the intensity of the external light, the nature of the chromophore, etc.

The lens can be rigid or foldable. The light absorbing material can be included in the IOL (contact lens, glasses), during the production of IOL or the IOL can be coated with light absorbing material.

The surface of the IOL (contact lens/glasses) can be modified to act as an light absorbing and anti-reflecting material as exposed to the external light.

The light absorbing material can be cross-linked with the IOL material

The chromophore in the polymer can be loose (inside the IOL or outside) or bound to the polymer. The lens after production can be encapsulated in a non-permeable thin layer of carbon (electron spattering, plasma spattering, nano-technology or other known methods) or other transparent molecules to retain the chromophore permanently inside the IOL and to prevent its exit. The external capsule retards or prevents actual or potential chromophore removal from the lens, e.g. leaching, diffusion, dissipation, etc., or to contain any loss that does or might occur.

The IOL can be implanted after a cataract extraction, or used as a contact lens over the existing crystalline lens in patients with loss of pigment (albinism), etc. or as an additive or after loss of a part of the Iris.

Because, in the light, the chromophore can create (by darkening) a new pupil (the center of IOL is spared), it creates a condition that the external objects (not only for object located in the far but also for those located at near), are always focused (pinhole effect) on the retina and eliminates spherical and chromatic aberration of the IOLs.

This concept can also be used in contact lenses and intra-corneal implants or reading glasses. Incorporating a potential pupil (spared from pigment-chemicals/material) in the IOLs contact lenses and glasses, eliminates the darkness that persists, when the entire lens is coated (as with standard transition lenses) and makes seeing or driving difficult, when e.g. a driver moves from the sun into a tunnel (light to darkness). The central area of the lens remains always substantially transparent and permits the driver to see objects in the dark area during the transition.

The above embodiments can be used for sunglasses. The lens can be plastic or glass. In both cases, the chromophore can be in the lens substance or coated. The uncoated portion can be circular of 2 mm to 15 mm or more in diameter. In one embodiment, the uncoated portion is circular of 2 mm to 5 mm diameter. The glass lenses are coated accordingly on their surface. The chromophore can be a UV absorber or respond to visible light only.

If the chromophore is responding to visible light for changing color to become dark and not UV, one can actually coat the central portion with a separate UV absorber without darkening the central area of the lenses while the peripheral portion will have photochromic properties such as silver chloride. Plastic photochromic lenses can have photochromic molecules oxazines and naphthopyrans for darkening effect. Lenses that darken in response to visible, rather than UV, light would avoid these issues. This implies that photochromic lenses are not transparent to UV light (they filter out UV light). In any case the central part of the lens has to be free of photochromic molecules that darken by exposure to visible or UV light.

The chromatic portion of the lens can be electrically stimulated to change the color as desired. However the uncoated central area remain transparent

In one use, the lens is implanted in or on a tissue (e.g., in the cornea, on the cornea, within a lens capsule, etc.). The lens may be encapsulated. The lens ameliorates glare and presbyopia by creating a pin hole in the visual axis in a lighted environment.

The coating material further comprises nanoparticles. The lenses in their peripheral part may also have nanoparticles that build solar cells. The solar cells may be used to power electrical systems for changing the pigments color and/or charge batteries, and may be located outside the glasses. The nanoparticles can have various functional abilities, such as a sensor that detects or measures wind speed, humidity, temperature, distance to an object, positioning (e.g., GPS), body temperature, etc

The nanoparticles may be in communication with another component. The other component may be located behind the ear. This component can have a wireless sender or receiver. The lens system may be a combination of electric and non-electric system.

In one embodiment, the diameter of the central area may be controlled. In one embodiment, the wearer controls the diameter of the central area. The above-described embodiments, e.g., sensor, may control a moveable diaphragm, may control darkening extent and rate, etc.

In one embodiment, the lens is for refractive correction. In one embodiment, the lens is to enhance vision. In one embodiment, the lens is cosmetic.

The lens is made of any transparent polymeric material having one or more light sensitive polymer with an increasing light-sensitive chromophore distribution from an area outside the center to the periphery. Stated in the alternative, the material has one or more light sensitive polymer with a decreasing light-sensitive chromophore distribution from a periphery to an area outside the center. In one embodiment, the increased chromophore distribution is uniform. In one embodiment, the increased chromophore distribution is not uniform. In one embodiment, there is a chromophore gradient. The distribution of chromophore is circular or substantially circular, or concentric or substantially concentric, around the center, with the center defining the visual axis.

In one embodiment, the frame may contain the chromophore-containing material, either the entire frame or one or more portions of the frame. The frame can be custom-molded and/or custom-fabricated for shape and/or size to minimize light from entering the eye. In one embodiment, the side frame can extend slightly beyond the plane of the iris. This embodiment minimizes light entry from the side. In one embodiment, the top frame can extend toward the forehead. This embodiment minimizes light entry from the top, simulating a half-goggle. In other embodiments, the nose bridge, ear piece, and/or other frame portions contain the chromophore-containing material. The frame may be fabricated such that the inventive lens can easily be inserted into (“pop in”) and removed from (“pop out”) the frame.

The application is not limited to the specific embodiments described and claimed. A person of ordinary skill in the art will recognize various modifications are possible.

What is claimed is:

1. An intraocular polymeric lens, the lens comprising
at least two concentric peripheral zones, and
an inner central zone defining a visual axis;
the polymeric material in the peripheral zones containing at least one light-activated chromophore that darkens when light-activated, the chromophore
in or on the lens polymeric material,
distributed in substantially concentric circles outward from the central area, and
increasing in concentration from the outer periphery of the central area to an outermost periphery of the outermost concentric circle;
the central area
lacking the chromophore, or
containing a chromophore that does not absorb visible light, or
containing a chromophore at a minimal concentration.

2. The lens of claim 1 where the chromophore concentration increases uniformly to the outermost periphery.

3. A refractive lens of claim 1.

4. A non-refractive lens of claim 1.

5. The lens of claim 1 where the central area diameter is at least 0.5 mm.

6. The lens of claim 5 where the central area diameter is up to 2 mm.

7. The lens of claim 5 where the central area diameter is up to 4 mm.

8. The lens of claim 5 where the central area diameter is up to 5 mm.

9. The lens of claim 5 where the central area diameter is up to 6 mm.

10. An encapsulated lens of claim 1.

11. The lens of claim 1 where the chromophore is selected from the group consisting of
a compound that selectively absorbs ultraviolet light;
a compound that selectively absorbs visible light;
a compound that selectively absorbs ultraviolet light and a compound that selectively absorbs visible light;
a compound that polarizes light; and
combinations thereof.

12. The lens of claim 1 in a frame.

13. The lens of claim 12 where the frame contains at least one light-activated chromophore.
